(19) United States
(12) Patent Application Publication
(10) Pub. No.: US 2026/0000331 A1
(43) Pub. Date: Jan. 1, 2026

(54) Apparatus And Method For Array Positioning

(71) Applicant: NOVOCURE GMBH, Baar (CH)

(72) Inventors: Andrew Duncan Pidgeon, Cambridge (GB); Niven Curtis-Woodcock, Cambridge (GB); Kathryn Anne Louise Farrell-Thomas, Cambridge (GB); Henry James Moorman, Cambridge (GB); Michael Robert George Jones, Cambridge (GB)

(21) Appl. No.: 19/252,106

(22) Filed: Jun. 27, 2025

Related U.S. Application Data
(60) Provisional application No. 63/665,833, filed on Jun. 28, 2024.

(51) Int. Cl. A61B 5/256; A61B 5/00

(52) U.S. Cl. CPC A61B 5/256; A61B 5/6831

(57) ABSTRACT

An apparatus for positioning at least one electrode array on a torso of a user is disclosed. The apparatus includes a strap that is configured to extend around the torso of the user, wherein the strap defines an inner surface that is configured to face the user. At least one alignment feature is provided on the inner surface of the strap, the at least one alignment feature indicating a position for securing the at least one electrode array to the inner surface of the strap.

### CROSS-REFERENCE TO RELATED APPLICATIONS

This application claims priority to and the benefit of the filing date of U.S. Provisional Patent Application No. 63/665,833, filed Jun. 28, 2024, the entirety of which is hereby incorporated by reference herein.

### FIELD

This disclosure relates to apparatuses, systems and methods for positioning electrode arrays on a user.

### BACKGROUND

Tumor Treating Fields (TTFields) therapy is a proven approach for treating tumors using alternating electric fields at frequencies between 50 kHz-1 MHz, more commonly, 100-500 kHz. In current commercial systems, the alternating electric fields are induced by electrode assemblies (e.g., arrays of capacitively coupled electrodes, also called transducer arrays or electrode arrays) placed on opposite sides of a target region of the subject's body. When an AC voltage is applied between opposing electrode arrays, an AC current is coupled through the electrode arrays and into the subject's body.

Proper positioning of electrode arrays relative to each other and a target region (e.g., a tumor) can affect performance of treatment. However, proper placement can be difficult, particularly when the subject is placing the electrode arrays on himself/herself. Thus, this difficulty can diminish the independence of the subject, requiring the subject to have another person (helper) position the electrode arrays. Accordingly, a way to assist a subject with properly positioning one or more electrode arrays is desirable.

### SUMMARY

TTFields are approved for the treatment of glioblastoma multiforme (GBM), and may be delivered, for example, via the OPTUNE® system (Novocure GmbH, Baar, Switzerland), which includes transducer arrays placed on the user's shaved head. More recently, TTFields therapy has been approved as a combination therapy with chemotherapy for malignant pleural mesothelioma (MPM) and non-small cell lung cancer (NSCLC), and may find use in treating tumors in other parts of the body. For example, recent studies have shown that TTFields treatment can reduce progression of metastases to the brain from non-small cell lung cancer (NSCLC).

Disclosed herein, in one aspect, an apparatus for positioning at least one electrode array on a torso of an individual (user). For example, the apparatus can be used to position the at least one electrode array on a torso of the user (e.g., a back of the user). The apparatus can include a strap that is configured to extend around the torso of the user, wherein the strap defines an inner surface that is configured to face the user. At least one alignment feature is provided on the inner surface of the strap, the at least one alignment feature indicating a position for securing the at least one electrode array to the inner surface of the strap.

Systems, methods, and kits using the apparatus are also disclosed herein.

### BRIEF DESCRIPTION OF THE DRAWINGS

FIG. 1 illustrates an apparatus being used for positioning at least one electrode array on a user.

FIG. 2 illustrates the apparatus of FIG. 1 in a partially opened position to show interior elements.

FIG. 3 illustrates an exemplary apparatus with a plug from a layer of foam removed to provide a recess that serves as an alignment feature of an exemplary apparatus as disclosed herein.

FIG. 4 illustrates placement of an electrode array into a recess of the exemplary apparatus of FIG. 3.

FIG. 5 illustrates removal of a release liner of the electrode array of FIG. 4.

FIG. 6 illustrates placement of the apparatus of FIG. 3 around a torso of a user.

FIG. 7 illustrates rotational alignment of the apparatus of FIG. 3 about the body of the user.

FIG. 8 illustrates removal of the apparatus of FIG. 3 from the user, leaving electrode arrays properly positioned on the user.

FIG. 9 is a partial cross-sectional view of an exemplary apparatus for positioning at least one electrode array as disclosed herein.

FIG. 10 is a partial cross-sectional view of the apparatus of FIG. 9 in tension.

FIG. 11 is a partial perspective view of an exemplary apparatus for positioning at least one electrode array as disclosed herein.

FIG. 12 is a partial front view of an exemplary apparatus for positioning at least one electrode array as disclosed herein.

FIG. 13 is a perspective view of an exemplary apparatus for positioning at least one electrode array as disclosed herein.

FIG. 14 is perspective view of the apparatus of FIG. 13 in an open position to show interior elements.

FIG. 15 is an inside view of the apparatus of FIG. 13 with electrode arrays positioned therein.

FIG. 16 shows electrode arrays placed on a user.

FIG. 17 shows the user wearing an exemplary apparatus for positioning at least one electrode array as disclosed herein.

FIG. 18 shows the apparatus of FIG. 17 once removed from the patient.

FIG. 19 shows application of electrode arrays to the apparatus of FIG. 17.

FIG. 20 shows tightening of the apparatus of FIG. 17 on the torso of the user.

FIG. 21 shows removal of release liner of the electrode arrays of FIG. 19.

FIG. 22 shows electrode arrays placed on a user.

Various embodiments are described in detail below with reference to the accompanying drawings, wherein like reference numerals represent like elements, and wherein descriptions of like elements may not be repeated for every embodiment, but may be considered to be the same if previously described herein.

### DESCRIPTION OF THE PREFERRED EMBODIMENTS

As discussed above, proper placement of electrode arrays can be difficult, particularly when the subject (a user) is placing the electrode arrays on himself/herself. This application relates to accurately positioning electrode arrays that may be used, e.g., for delivering TTFields to a subject's body (for example, the lungs) and treating one or more cancers or tumors located in the subject's body (e.g., the lungs). The subject may then reproduce the accurate positioning of the electrode arrays for future application of the arrays on the torso of the subject. For example, the disclosed apparatus can be used to position at least one electrode array on the back of the subject. Optionally, the disclosed apparatus can be used to position at least one electrode array on the back of the subject and the front of the subject. For example, the disclosed apparatus can be used to position an electrode array on each side of the chest and an electrode array on each side of the back. In this way, a pair of electrode arrays can be positioned on opposite sides of each lung.

The present invention can be understood more readily by reference to the following detailed description, examples, drawings, and claims, and their previous and following description. However, it is to be understood that this invention is not limited to the specific apparatuses, devices, systems, and/or methods disclosed unless otherwise specified, and as such, of course, can vary.

Any combination of the elements described herein in all possible variations thereof is encompassed by the invention unless otherwise indicated herein or otherwise clearly contradicted by context. Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

As used in the specification and the appended claims, the singular forms “a,” “an,” and “the” include plural referents unless the context clearly dictates otherwise. Thus, it is contemplated that disclosure of a singular form of an element can provide support for embodiments in which only a single such element is provided, as well as support for embodiments in which a plurality of such elements are provided.

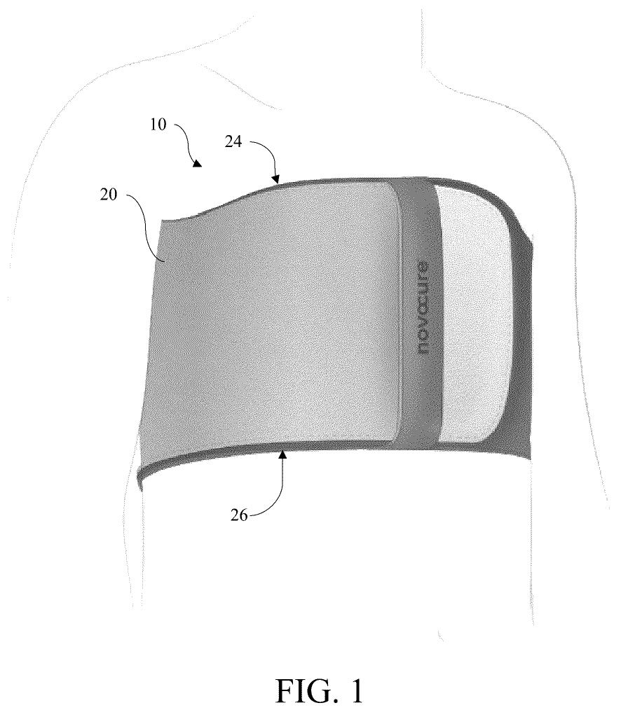

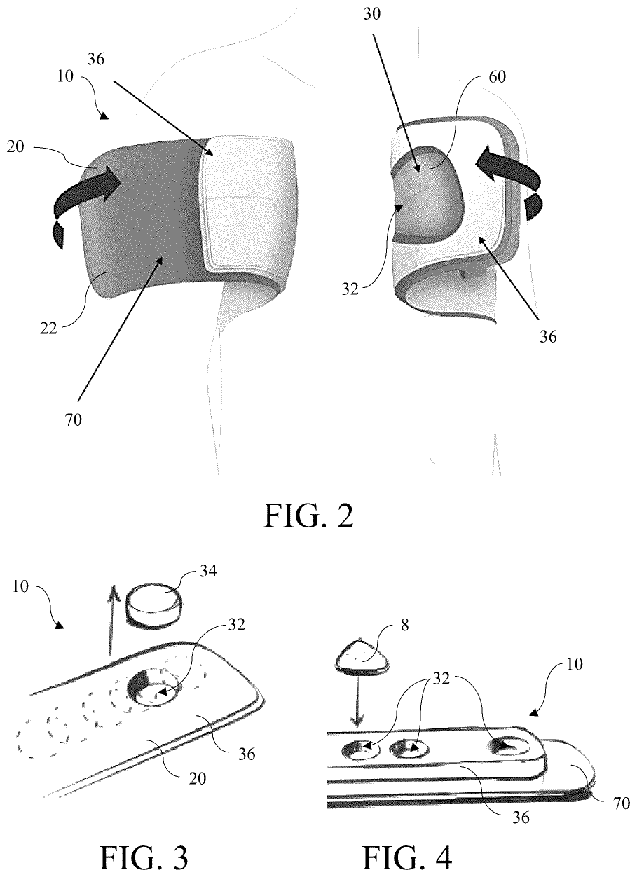

Disclosed herein are apparatuses for positioning at least one electrode array in a predetermined (e.g., optimal) position on a torso of a user. FIG. 1 illustrates an apparatus 10 being used for positioning at least one electrode array on a torso of a user. Referring also to FIG. 2, the apparatus 10 comprises a strap 20 that is configured to extend around the torso of the user. The strap 20 has an inner surface 22 that is configured to face the user. At least one alignment feature 30 can be provided on the inner surface 22 of the strap 20. The at least one alignment feature 30 can indicate a position for securing the at least one electrode array to the inner surface of the strap.

The strap 20 can have an upper edge 24 and a lower edge 26. In exemplary aspects, the at least one alignment feature 30 can be spaced from the upper edge 24 or the lower edge 26 of the strap 20 so that a vertical position of the alignment feature relative to the user is determined when the upper edge or the lower edge is aligned with a bodily feature of the user. For example, the upper edge 24 can be configured so the at least one alignment feature 30 can be spaced from the upper edge 24 of the strap 20 so that when further upward movement of the strap 20 is limited by the armpits of the user, the vertical position of the one alignment feature 30 (and, accordingly, the position of an electrode array aligned relative thereto) is determined.

The apparatus 10 can comprise at least one attachment element 60 on the inner surface 22 of the strap 20. The at least one attachment element 60 can be configured to releasably retain the electrode array 8 (FIG. 4) against the strap 20. For example, the at least one attachment element 60 can comprise hook or loop material, a snap-fastener, or an adhesive, or combinations thereof. It is contemplated that the attachment element(s) 60 can have a lower release force than the skin-facing adhesive or gel on the electrode array so that as the apparatus 10 is removed from the user, the electrode array can remain attached to the user.

Optionally, the at least one alignment feature 30 can comprise a marking. For example, the marking can comprise a grid. Optionally, in these aspects, the user can be provided with additional information for positioning the at least one electrode array relative to the grid. For example, the additional information can include written instructions (e.g., grid coordinates) and/or an illustration indicating the location for each electrode array of the at least one electrode array. In other aspects, the marking can be specific to the user. For example, the marking can include a user-specific marking printed on the strap 20 for indicating a location for the at least one electrode array. Optionally, in these aspects, the marking can correspond to a center of an electrode array or an outer perimeter of an electrode array. For example, the marking(s) may trace a portion of, or an entirety of, an outer perimeter of each electrode array of the at least one electrode array.

In other aspects, the at least one alignment feature 30 can comprise an attachment element 60. For example, each attachment element 60 can be in a predetermined position on the strap 20 that is indicative of a location for the electrode array.

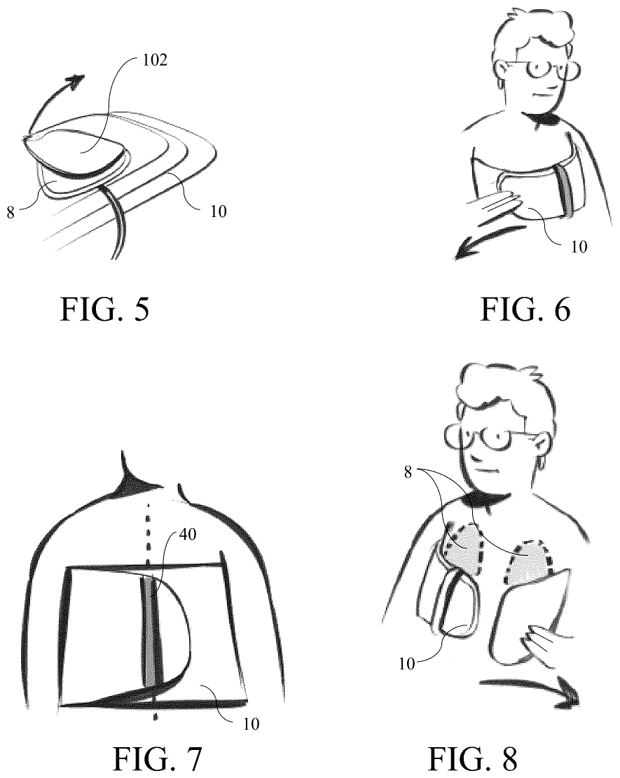

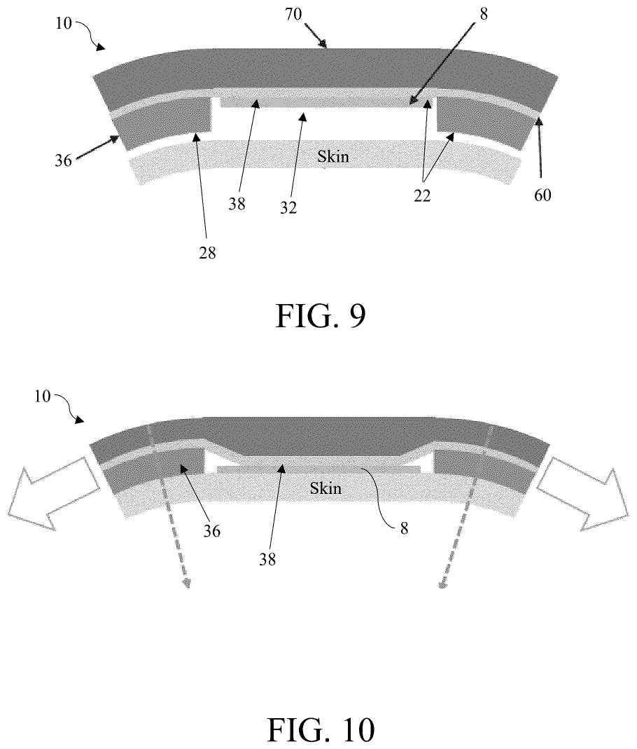

Referring to FIGS. 2-4 and 9-10, in other aspects, the at least one alignment feature 30 can comprise at least one recess 32 in the inner surface 22 of the strap 20. Each recess 32 can be configured to receive a respective electrode array 8 of the at least one electrode array therein. In exemplary aspects, the strap 20 can define a plurality of recesses 32. Optionally, one or more of the plurality of recesses 32 can have a plug 34 therein. For example, optionally, as illustrated in FIG. 3, the strap 20 can comprise a plurality of recesses 32 that may or may not overlap. A plug 34 can be removed from the plurality of recesses 32 to expose the recess. In exemplary aspects, the plug 34 can be integrally formed within the strap, connected to a remainder of the strap, partially separated by a plurality of perforations. The perforations can permit the plug 34 to be fully separated from the strap to expose the plug. For example, in exemplary aspects, the plug(s) 34 can comprise foam. In further aspects, the strap 20 can comprise a layer of foam 36, and the plugs 34 can be portions that are partially (e.g., by perforations) or fully separated from the layer of foam. In other aspects, the plugs 34 can be secured in the strap via a compression fit and/or by at least one fastener (e.g., hook and loop fastener, snap, or adhesive). In exemplary aspects, and with reference to FIG. 9, the plug can be removed to expose an attachment element 60 (e.g., underlying layer of hook or loop fastener). FIGS. 5-8 show operational aspects of the apparatus, discussed further below.

Referring to FIGS. 9-10, optionally, the strap 20 can comprise a flexible material 70 positioned outwardly of the at least a portion of the strap that is configured to compress (e.g., the layer of foam 36). Optionally, the flexible material 70 can be inelastic. In other aspects, the flexible material 70 can be compressible. In exemplary aspects, an attachment element 60 can be positioned between the flexible material 70 and the portion of the strap 20 that is configured to compress. For example, the attachment element 60 can comprise hook or loop fastener. Accordingly, the plug 34 (FIG. 4) can be removed to expose the hook or loop material and to provide the recess 32 in a desired location.

Referring to FIGS. 9-10, the inner surface 22 of the strap 20 can comprise a contact surface 28 configured to face skin of the torso of the user. The recess 32 can comprise a bottom surface 38 spaced radially outwardly from the contact surface 28 (e.g., radially outwardly from a plane of the contact surface when the contact surface is flat). Upon tightening of the strap 20 against the torso, at least a portion of the strap can be configured to compress so that the bottom surface 38 of the recess 32 moves radially inwardly relative to the contact surface of the strap. For example, the layer of foam 36 can compress. In this way, the electrode array 8 can be drawn against the skin of the user.

The portion of the strap 20 configured to compress (e.g., the layer of foam 36) can prevent contact of the at least one electrode array 8 with the torso of the user prior to tightening the strap. For example, in an uncompressed state, said portion of the strap (e.g., the layer of foam 36) can space the electrode array 8 from the user (FIG. 9). Tightening of the strap against the torso can bring a skin-facing adhesive or gel on the electrode array 8 into contact with the torso of the user (FIG. 10).

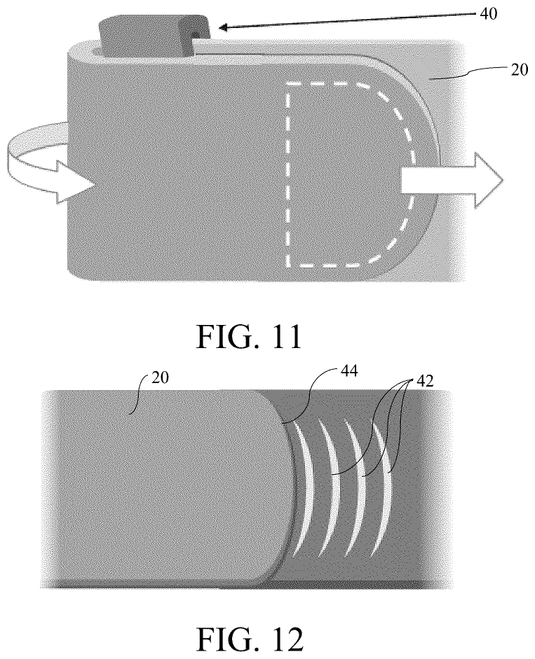

Referring to FIG. 11, the apparatus 10 can further comprise a belt loop 40. The strap 20 can be configured to extend through the belt loop 40. In some aspects, the belt loop 40 can permit a user to pull thereagainst to apply a greater leverage when tightening down the strap 20. In some aspects, the belt loop 40 can be coupled to the strap 20 so that the belt loop serves as a reference point relative to rotational position about the torso of the user. For example, as illustrated in FIG. 7, the belt loop 40 can be aligned with a center of a chest (e.g., sternum) of a user. Accordingly, the belt loop 40 can be used as an indicator for rotational alignment of the apparatus 10 relative to the user. In this way, the apparatus 10 and, therefore, the electrode array(s) 8 associated therewith, can be rotationally positioned about the body of the user.

Referring to FIG. 12, in some aspects, the strap 20 can comprise at least one marking 42 indicative of a tightness of the strap 20. For example, the marking(s) 42 can be compared to an end 44 of the strap 20. Accordingly, a tightness of the strap 20 can be measured. In some aspects, the at least one marking can comprise a plurality of markings. In exemplary aspects, a particular user can be instructed to tighten the strap 20 to a particular marking 42. In this way, based on the size of the user, a particular marking 42 can be selected to be associated with the strap being pulled sufficiently tightly to effectively apply the one or more electrodes to the user.

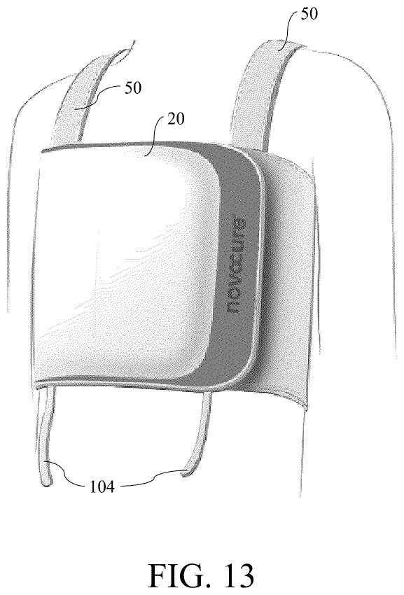

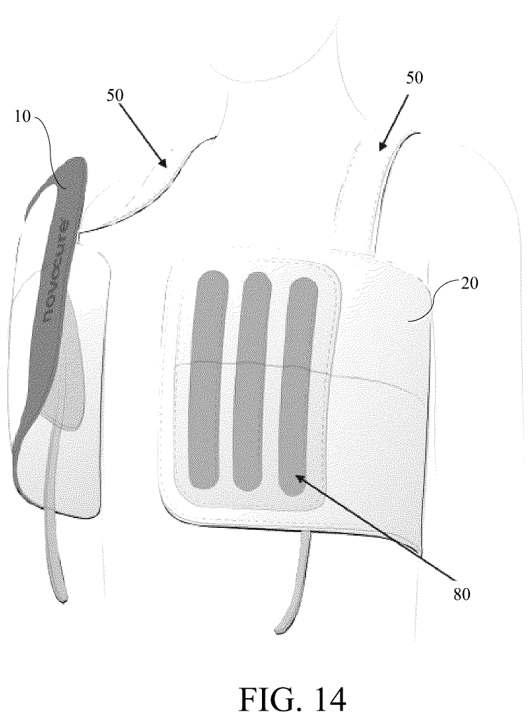

Referring to FIGS. 13-14, in some aspects, the apparatus 10 can comprise at least one shoulder strap 50 coupled to the strap 20. Each shoulder strap 50 can be configured to extend over a respective shoulder of the user. Optionally, each shoulder strap 50 can be adjustable to determine a height of the strap 20. Further, by being adjustable, the shoulder straps 50 can permit the apparatus 10 to be used with users of different sizes. Optionally, the shoulder straps 50 can be used to locate the apparatus 10 (e.g., by aligning relative to body features such as moles or freckles). In some aspects, and with reference to FIG. 14, the apparatus 10 can be in the form of a wearable vest. For example, the wearable vest can comprise a body that is configured to surround the torso and extend over shoulders of the user. The body can further define arm holes.

Optionally, the apparatus 10 can comprise a fastener 80, such as hook and loop fastener to retain the apparatus tightly against the torso of the user.

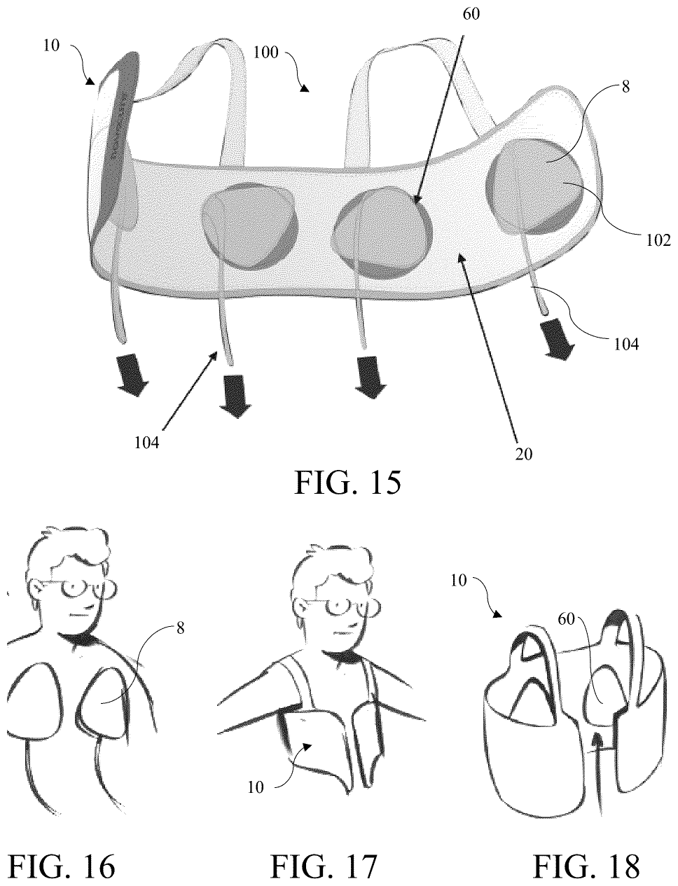

Referring to FIG. 15, a system 100 can comprise an apparatus 10 as disclosed herein and at least one electrode array. The at least one alignment feature 30 (FIG. 2) of the apparatus 10 can indicate a position for securing the at least one electrode array to the inner surface of the strap.

In some aspects, each electrode array 8 can comprises a release liner 102. In some optional aspects, the release liner 102 can comprise a tab 104 having a length sufficient to extend outwardly from the strap when the electrode array is secured to the inner surface of the strap. For example, the tab 104 of the release liner 102 can be at least 4 inches long, at least 6 inches, long, or at least 8 inches long.

A method for positioning at least one electrode array 8 on a torso of a user can include securing the at least one electrode array to the inner surface 22 of the strap 20 of the apparatus 10. The strap 20 can be wrapped around the torso of the user. The strap 20 can be rotationally positioned around the torso of the user. In various aspects, the strap 20 can be rotationally positioned by aligning a marking on the strap with a particular part of the body (e.g., a center of a chest), aligning the belt loop 40 with a particular part of the body, or by inserting a respective arm through the at least one shoulder strap 50 (where provided). The strap 20 can be tightened down against the torso of the user so that the at least one electrode array 8 contacts skin of the user.

In aspects in which the at least one alignment feature 30 comprises at least one recess 32, each recess being configured to receive a respective electrode array 8, the method can comprise positioning the respective electrode array within each recess of the at least one recess. Optionally, in these aspects, the strap 20 can be tightened so that a thickness of at least a portion of the strap compresses and the at least one electrode array 8 is pulled into contact with skin of the user.

Referring to FIGS. 11-12, in aspects in which the apparatus 10 comprises at least one marking 42 indicative of a tightness of the strap 20, the strap can be tightened so that the at least one electrode array 8 contacts the skin of the user. In such aspects, the method can further comprise tightening the strap based on a position of the end 44 of the strap relative to the at least one marking 42. For example, the strap can be pulled to a prescribed marking 42.

In aspects in which each electrode array 8 comprises a release liner 102 having a tab 104, the method can comprise pulling the tab to release the release liner with the strap 20 wrapped around the torso of the user and at least a portion of the electrode array in contact with the skin of the user.

In one example, and with reference to FIGS. 3-8, a medical professional can remove a portion of the layer of foam 36 to form a recess 32 or a plurality of recesses (FIG. 3). Optionally, the medical professional can use pre-formed perforations to remove the portion of the layer of foam 36. Each recess 32 can serve as an alignment feature 30 for each electrode array 8 (FIG. 4). The user can position an electrode array 8 in each recess 32. The user can remove the release liner 102 from the electrode array 8 (FIG. 5). The user can wrap the apparatus 10 around her torso (FIG. 6). The user can align the belt loop 50 with her sternum (FIG. 7). The user can pull the strap tightly to compress the layer of foam 36 so that the electrode array(s) 8 stick to the skin of the user. The user can remove the strap, leaving the arrays correctly positioned on the skin of the torso of the user (FIG. 8).

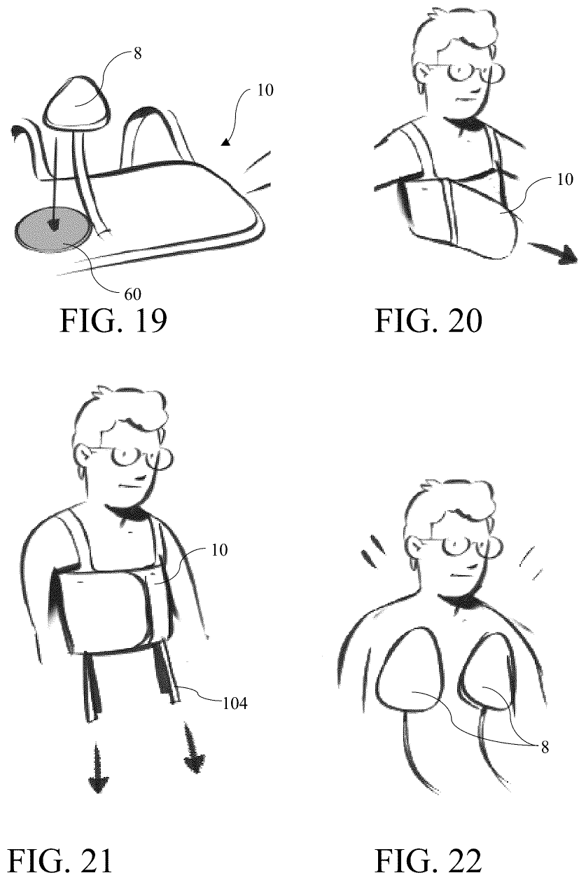

In one example, and with reference to FIGS. 16-22, a medical professional can apply one or more electrode arrays 8 to a user in proper locations on the torso of the user (FIG. 16). Each electrode array 8 can comprise a respective applicator module (e.g., attachment element 60). Referring to FIGS. 17-18, the apparatus 10 can be placed over the user and properly fitted. When the apparatus 10 is removed, the applicator module(s) can remain coupled to the apparatus, thereby serving as alignment feature(s) 30, indicating the location for attaching electrode arrays in the future. Referring to FIG. 19, the user (optionally, without professional oversight) can place the electrode arrays on the applicator modules. The user can place the apparatus on her body (FIG. 20). She can pull the tabs 104 to remove the release liner 102 and expose the skin-facing adhesive or gel on the electrode array to the skin of the user (FIG. 21). The user can press on the applicator and can wait a predetermined duration (e.g., at least one minute) before removing the apparatus, leaving the electrode array(s) 8 attached in the proper locations (FIG. 22).

### EXEMPLARY ASPECTS

In view of the described apparatuses, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: An apparatus for positioning at least one electrode array on a torso of a user, the apparatus comprising:
a strap that is configured to extend around the torso of the user, wherein the strap defines an inner surface that is configured to face the user; and
at least one alignment feature provided on the inner surface of the strap, the at least one alignment feature indicating a position for securing the at least one electrode array to the inner surface of the strap.

Aspect 2: The apparatus of aspect 1, wherein the strap has an upper edge and a lower edge, wherein the at least one alignment feature is spaced from the upper edge or the lower edge of the strap so that a vertical position of the alignment feature relative to the user is determined when the upper edge or the lower edge is aligned with a bodily feature of the user.

Aspect 3: The apparatus of aspect 1 or aspect 2, wherein the at least one alignment feature comprises a marking.

Aspect 4: The apparatus of any one of the preceding aspects, wherein the at least one alignment feature comprises at least one recess in the inner surface of the strap, wherein each recess of the at least one recess is configured to receive a respective electrode array of the at least one electrode array therein.

Aspect 5: The apparatus of aspect 4, wherein the inner surface of the strap comprises a contact surface configured to face skin of the torso of the user, wherein the recess comprises a bottom surface spaced radially outwardly from a plane of the contact surface, wherein, upon tightening of the strap against the torso, at least a portion of the strap is configured to compress so that the bottom surface of the recess moves radially inwardly relative to the plane of the contact surface of the strap.

Aspect 6: The apparatus of aspect 4 or aspect 5, wherein the at least one recess comprises a plurality of recesses.

Aspect 7: The apparatus of aspect 6, wherein the strap comprises at least one removable plug, each removable plug of the at least one removable plug positioned within a respective recess of the plurality of recesses.

Aspect 8: The apparatus of any one of aspects 5-7, wherein at least a portion of the strap that is configured to compress is configured to prevent contact of the at least one electrode array with the torso of the user prior to tightening of the strap.

Aspect 9: The apparatus of aspect 8, wherein tightening of the strap against the torso is configured to bring a skin-facing adhesive or gel on the electrode array into contact with the torso of the user.

Aspect 10: The apparatus of aspect 9, wherein, upon tightening of the strap against the torso, the at least a portion of the strap is configured to compress toward the torso of the user.

Aspect 11: The apparatus of any one of aspects 5-10, wherein the at least a portion of the strap that is configured to compress comprises a layer of foam.

Aspect 12: The apparatus of any one of aspects 5-11, wherein the strap further comprises a flexible material positioned outwardly of the at least a portion of the strap that is configured to compress the at least a portion of the strap against the user when tension is applied thereto.

Aspect 13: The apparatus of any one of the preceding aspects, further comprising a belt loop, wherein the strap is configured to extend through the belt loop.

Aspect 14: The apparatus of aspect 13, wherein the belt loop is positioned as a reference point relative to rotational position about the torso of the user.

Aspect 15: The apparatus of any one of the preceding aspects, further comprising at least one marking indicative of a tightness of the strap.

Aspect 16: The apparatus of aspect 15, wherein the at least one marking comprises a plurality of markings.

Aspect 17: The apparatus of any one of the preceding aspects, further comprising at least one shoulder strap, wherein each shoulder strap of the at least one shoulder strap is configured to extend over a respective shoulder of the user.

Aspect 18: The apparatus of aspect 17, wherein the apparatus is in the form of a wearable vest.

Aspect 19: The apparatus of any one of the preceding aspects, further comprising at least one attachment element on the inner surface of the strap, wherein the at least one attachment element is configured to releasably retain the electrode array against the strap.

Aspect 20: The apparatus of aspect 19, wherein the at least one attachment element comprises hook or loop material, a snap-fastener, or an adhesive.

Aspect 21: A system comprising:
an apparatus as in any one of aspects 1-20; and
at least one electrode array,
wherein the at least one alignment feature of the apparatus indicates a position for securing the at least one electrode array to the inner surface of the strap.

Aspect 22: The system of aspect 21, wherein each electrode array comprises a release liner, the release liner comprising a tab having a length sufficient to extend outwardly from the strap when the electrode array is secured to the inner surface of the strap.

Aspect 23: A method for positioning at least one electrode array on a torso of a user, the method comprising:
securing at least one electrode array to the inner surface of the strap of the apparatus of any one of aspects 1-20;
wrapping the strap around the torso of the user;
optionally, rotationally positioning the strap around the torso of the user; and
tightening the strap so that the at least one electrode array contacts skin of the user.

Aspect 24: The method of aspect 23, wherein the at least one alignment feature comprises at least one recess, wherein each recess of the at least one recess is configured to receive a respective electrode array of the at least one electrode array therein, the method comprising positioning the respective electrode array within each recess of the at least one recess.

Aspect 25: The method of aspect 24, further comprising tightening the strap so that a thickness of at least a portion of the strap compresses and the at least one electrode array is pulled into contact with skin of the user.

Aspect 26: The method of any one of aspects 23-25, wherein the apparatus further comprises a belt loop, wherein rotationally positioning the strap around the user comprises aligning the belt loop with a center of a chest of the user.

Aspect 27: The method of any one of aspects 23-26, wherein the apparatus comprises at least one marking indicative of a tightness of the strap, wherein tightening the strap so that the at least one electrode array contacts the skin of the user comprises tightening the strap based on a position of an end of the strap relative to the at least one marking.

Aspect 28: The method of any one of aspects 23-27, wherein each electrode array of the at least one electrode array comprises a release liner, the release liner comprising a tab having a length that extends outwardly from the strap when the electrode array is positioned on the inner surface of the strap, the method comprising pulling the tab to release the release liner with the strap wrapped around the torso of the user and at least a portion of the electrode array in contact with the skin of the user.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. An apparatus for positioning at least one electrode array on a torso of a user, the apparatus comprising:
a strap that is configured to extend around the torso of the user, wherein the strap defines an inner surface that is configured to face the user; and
at least one alignment feature provided on the inner surface of the strap, the at least one alignment feature indicating a position for securing the at least one electrode array to the inner surface of the strap.

2. The apparatus of claim 1, wherein the strap has an upper edge and a lower edge, wherein the at least one alignment feature is spaced from the upper edge or the lower edge of the strap so that a vertical position of the alignment feature relative to the user is determined when the upper edge or the lower edge is aligned with a bodily feature of the user.

3. The apparatus of claim 1, wherein the at least one alignment feature comprises a marking.

4. The apparatus of claim 1, wherein the at least one alignment feature comprises at least one recess in the inner surface of the strap, wherein each recess of the at least one recess is configured to receive a respective electrode array of the at least one electrode array therein.

5. The apparatus of claim 4, wherein the inner surface of the strap comprises a contact surface configured to face skin of the torso of the user, wherein the recess comprises a bottom surface spaced radially outwardly from a plane of the contact surface, wherein, upon tightening of the strap against the torso, at least a portion of the strap is configured to compress so that the bottom surface of the recess moves radially inwardly relative to the plane of the contact surface of the strap.

6. The apparatus of claim 5, wherein at least a portion of the strap that is configured to compress is configured to prevent contact of the at least one electrode array with the torso of the user prior to tightening of the strap.

7. The apparatus of claim 6, wherein tightening of the strap against the torso is configured to bring a skin-facing adhesive or gel on the electrode array into contact with the torso of the user.

8. The apparatus of claim 5, wherein the strap further comprises a flexible material positioned outwardly of the at least a portion of the strap that is configured to compress the at least a portion of the strap against the user when tension is applied thereto, and, optionally, wherein the at least a portion of the strap that is configured to compress comprises a layer of foam.

9. The apparatus of claim 1, further comprising a belt loop, wherein the strap is configured to extend through the belt loop.

10. The apparatus of claim 9, wherein the belt loop is positioned as a reference point relative to rotational position about the torso of the user.

11. The apparatus of claim 1, further comprising at least one attachment element on the inner surface of the strap, wherein the at least one attachment element is configured to releasably retain the electrode array against the strap.

12. The apparatus of claim 11, wherein the at least one attachment element comprises hook or loop material, a snap-fastener, or an adhesive.

13. The apparatus of claim 1, further comprising at least one shoulder strap, wherein each shoulder strap of the at least one shoulder strap is configured to extend over a respective shoulder of the user.

14. The apparatus of claim 13, wherein the apparatus is in the form of a wearable vest.

15. A system comprising:
an apparatus for positioning at least one electrode array on a torso of a user, the apparatus comprising:
a strap that is configured to extend around the torso of the user, wherein the strap defines an inner surface that is configured to face the user; and
at least one alignment feature provided on the inner surface of the strap, the at least one alignment feature indicating a position for securing the at least one electrode array to the inner surface of the strap; and
at least one electrode array,
wherein the at least one alignment feature of the apparatus indicates a position for securing the at least one electrode array to the inner surface of the strap.

16. The system of claim 15, wherein each electrode array comprises a release liner, the release liner comprising a tab having a length sufficient to extend outwardly from the strap when the electrode array is secured to the inner surface of the strap.

17. A method for positioning at least one electrode array on a torso of a user, the method comprising:
securing at least one electrode array to an inner surface of a strap of an apparatus comprising:
the strap, wherein the strap is configured to extend around the torso of the user, wherein the strap defines the inner surface, wherein the inner surface is configured to face the user; and
at least one alignment feature provided on the inner surface of the strap, the at least one alignment feature indicating a position for securing the at least one electrode array to the inner surface of the strap;
wrapping the strap around the torso of the user;
optionally, rotationally positioning the strap around the torso of the user; and
tightening the strap so that the at least one electrode array contacts skin of the user.

18. The method of claim 17, wherein the at least one alignment feature comprises at least one recess, wherein each recess of the at least one recess is configured to receive a respective electrode array of the at least one electrode array therein, the method comprising positioning one or more respective electrode array of the at least one electrode array within one or more respective recess of the at least one recess.

19. The method of claim 18, further comprising tightening the strap so that a thickness of at least a portion of the strap compresses and the at least one electrode array is pulled into contact with skin of the user.

20. The method of claim 17, wherein the apparatus further comprises a belt loop, wherein rotationally positioning the strap around the user comprises aligning the belt loop with a designated location of the user's body, optionally aligning the belt loop with a center of a chest of the user.
